(19) United States
(12) Patent Application Publication
(10) Pub. No.: US 2026/0317677 A1
(43) Pub. Date: Oct. 15, 2026

(54) CELL PEELING DEVICE AND CELL PEELING METHOD

(71) Applicant: Hitachi, Ltd., Tokyo (JP)

(72) Inventors: Kota SASAKI, Tokyo (JP); Yuusuke KOURAI, Tokyo (JP); Mutsumi SUZUKI, Tokyo (JP)

(73) Assignee: Hitachi, Ltd., Tokyo (JP)

(21) Appl. No.: 18/853,477

(22) PCT Filed: Feb. 15, 2023
(86) PCT No.: PCT/JP2023/005095

§ 371 Date: Oct. 2, 2024

(30) Foreign Application Priority Data
Jun. 20, 2022 (JP) 2022-098790

(51) Int. Cl. C12M 1/26; C12M 1/36

(52) U.S. Cl. CPC C12M 33/08; C12M 41/48

(57) ABSTRACT

A cell peeling device and method, for peeling a cell aggregate from a culture vessel in a non-contact manner. The cell peeling device is aimed at peeling a cultured cell aggregate from a culture vessel in which the cell aggregate was cultured, the device includes: at least one ultrasonic probe for irradiating the cell aggregate with ultrasonic beam having a beam diameter smaller than a size of the cell aggregate, from the outside of the culture vessel; at least one move mechanism for changing relative position between the culture vessel and the ultrasonic probe; an edge recognition unit for recognizing an edge of the cell aggregate; and a control unit for controlling the move mechanism and the ultrasonic probe, by calculating a trajectory along which the ultrasonic probe moves from the edge of the cell aggregate, and an output level of the ultrasonic beam at a point on the trajectory.

### TECHNICAL FIELD

The present invention relates to a cell peeling device and a cell peeling method for peeling a specific cell aggregate, including cell sheet, organoid (may be simply referred to as “cell aggregate”, hereinafter) or the like in a culture vessel, from a culture surface, with the aid of ultrasonic wave.

### BACKGROUND ART

Treatment with use of cultured cells has been put into practical use, for treatment with the aid of regenerative medicine. Cells proliferate in specific cell culture vessels including cell culture dish, cell culture plate, multi-well plate, microtiter plate, flask, multi-cell culture flask, and multi-layer cell culture vessel (may be simply referred to as “culture vessel”, hereinafter). The cells during culture grow while adhered to a bottom face of the culture vessel. Grown cells form a cell aggregate typically in the form of cell sheet, to be used for example in treatment.

An exemplary method for peeling the cells adhered to the culture vessel from the vessel has been proposed (PTL 1), which is a technique that relies upon mechanical vibration caused by a piezoelectric element adhered to the cell culture vessel. PTL 1 describes that the mechanical vibration is applied uniformly over the cultured cells, to peel the entire range of the cell sheet from the culture vessel in a non-contact manner.

### CITATION LIST

### Patent Literature

PTL 1: WO 2019/044804 A1

### SUMMARY OF INVENTION

### Technical Problem

The cell aggregate, while being adhered to the culture vessel, is however not uniform in terms of cell proliferation. The technique described in PTL 1 has therefore been suffered from that an effort of evenly peeling the cell sheet is time-consuming and inefficient.

The present invention has been made in view of the above problem, and an object thereof is to provide a cell peeling device and a cell peeling method, capable of peeling a cell aggregate from a culture vessel at high speed in a non-contact manner.

### Solution to Problem

Aimed at solving the aforementioned problem, a cell peeling device according to the present invention is a cell peeling device that peels a cell aggregate from a culture vessel, the cell peeling device includes: at least one ultrasonic probe structured to irradiate the cell aggregate with ultrasonic beam having a beam diameter smaller than a size of the cell aggregate, from an outside of the culture vessel; at least one move mechanism structured to change relative position between the culture vessel and the ultrasonic probe; an edge recognition unit capable of recognizing an edge of the cell aggregate; and a control unit structured to control the move mechanism and the ultrasonic probe, by calculating a trajectory along which the ultrasonic probe moves from the edge of the cell aggregate, and an output level of the ultrasonic beam at a point on the trajectory.

### Advantageous Effects of Invention

The present invention can provide a cell peeling device and a cell peeling method, capable of peeling a cell aggregate from a culture vessel at high speed in a non-contact manner. Further features related to the present invention will become apparent from the description of the present specification and the accompanying drawings. Problems, structures, and effects other than those described above will be clarified by the following description of embodiments.

### BRIEF DESCRIPTION OF DRAWINGS

FIG. 1 is a schematic structural diagram of a cell peeling device according to a first embodiment.

FIG. 2A is a schematic diagram illustrating an exemplary moving route of an ultrasonic beam.

FIG. 2B is a schematic diagram illustrating an exemplary moving route of an ultrasonic beam.

FIG. 3 is an explanatory diagram illustrating an exemplary electric signal.

FIG. 4 is a flowchart for describing details of a cell peeling method according to the first embodiment.

FIG. 5 is a schematic diagram for describing stress that exerts on cells due to the cell peeling method in Examples.

FIG. 6 is a schematic diagram for describing stress exerted on the cells due to the cell peeling method of Comparative Example.

FIG. 7 is a schematic structural diagram of a cell peeling device according to a second embodiment.

### DESCRIPTION OF EMBODIMENTS

Embodiments of the present invention will be described below, based on the attached drawings.

In this embodiment, the cell aggregate being adhered to the inner wall face of the culture vessel will be peeled. The cell peeling device of this embodiment may be used as a part of a cell culture apparatus. The cell peeling method of this embodiment herein will be described, while exemplifying a method of culturing adherent cells by forming a cell culture medium in a usual cell culture dish. An applicable range of the present invention is, however, not limited thereto. This embodiment is applicable to various types of adherent cells that can be cultured, without being limited to specific cells. In addition, the culture medium applicable to the cell culture in this embodiment may either be serum-free culture medium, or serum-containing culture medium.

This embodiment will be described with reference to a system with a cell aggregate, kept immersed in a medium in a culture vessel, and kept adhered on a bottom inner face which is an inner wall face of the culture vessel, in which ultrasonic wave is transmitted upwards, that is, in a direction from the bottom outer face to the bottom inner face of the culture vessel. This embodiment is, however, not limited to this system, or may be aimed at any system in which the ultrasonic wave coming from the lateral side or from above of the culture vessel is transmitted through the medium.

The cell peeling device and the method of this embodiment make a touch of, or close contact of an ultrasonic prove having a piezoelectric element, with the outer face of the culture vessel, which is typically the bottom outer face, and transmit freely controlled ultrasonic wave through the culture vessel to the cell aggregate in the culture vessel. The ultrasonic probe and the outer face of the culture vessel in this case may be contacted, while placing in between an acoustic coupler for efficiently transmitting the ultrasonic wave, or a matching layer typically formed of water, silicon rubber, or fluororubber.

The cell peeling device and the method of this embodiment transmit the ultrasonic wave at a certain level or higher output level from the ultrasonic probe, thereby applying acoustic radiation pressure to the cell aggregate in the culture vessel. The transmitted ultrasonic wave can produce, on the route thereof, an acoustic flow. The cell aggregate may be peeled from the inner wall face of the culture vessel, upon application of upward force ascribed to the acoustic radiation pressure and/or the acoustic flow towards the cell aggregate.

The freely controlled ultrasonic wave in this embodiment is exemplified by ultrasonic wave having electronically controlled output level, including size of ultrasonic beam (beam diameter) at the boundary between the cell aggregate and the inner wall face of the culture vessel, intensity of ultrasonic beam (output density), transmission time of ultrasonic beam (duty ratio, repetition interval). The ultrasonic beam corresponds to a band-shaped or rod-shaped zone formed by the ultrasonic wave transmitted from the piezoelectric element.

The intensity of the ultrasonic beam is controllable, typically by the methods below. For example, the intensity of the ultrasonic beam is controllable by changing voltage intensity applied to the piezoelectric element of the ultrasonic probe, or by changing the wave number of waveform of voltage to be applied. For the ultrasonic probe having a plurality of piezoelectric elements, voltage may be applied to all of, or a selected part of the piezoelectric elements.

The transmission time of the ultrasonic beam is controllable, typically by the methods below. That is, it suffices to change the wave number or application time of the voltage to be applied, at a specific frequency, to the piezoelectric element of the ultrasonic probe. Note that the aforementioned specific frequency is given by size and material of the piezoelectric element, and is freely determined according to the piezoelectric element. The methods for controlling the intensity and transmission time of the ultrasonic beam, although described as above, are not limited to the aforementioned methods.

Aimed at recognizing the edge of the cell aggregate during the culture, the device may be equipped with an edge recognition unit having been widely used for cell observation, such as optical camera, optical microscope, or phase contrast microscope. The edge recognition unit observes shape or coordinates of the edge or the like of the cell aggregate, and stores them in an edge status database. A probe trajectory calculation unit calculates a trajectory of the ultrasonic probe, with reference to the coordinates of the edge of the cell aggregate stored in the edge status database.

A beam adjustment calculation unit refers to the shape and coordinates of the edge stored in the edge status database, and calculates output levels of the ultrasonic beam, such as the intensity or transmission time thereof, to determine the transmission condition of the ultrasonic wave. The probe trajectory calculation unit and the beam adjustment calculation unit may refer to the coordinates of the edge preliminarily stored in the edge status database, corresponding to shape of the culture vessel and the number of days of culture.

The move mechanism is structured to move the ultrasonic probe to any desired point, so as to trace the trajectory determined by the probe trajectory calculation unit. The move mechanism may alternatively move both the culture vessel and the ultrasonic probe, or may move the culture vessel relative to the ultrasonic probe. A voltage application unit is structured to apply voltage to the piezoelectric element according to the transmission condition of the ultrasonic wave determined by the beam adjustment calculation unit, thereby causing transmission of the ultrasonic wave, and peeling of the cell aggregate having been adhered to the inner wall face of the culture vessel.

The cell peeling device of this embodiment is structured to recognize the edge of the cell aggregate, to adjust the beam intensity based on the shape and coordinate data of the cell aggregate, and to locally transmit the ultrasonic wave from the outside of the culture vessel, thereby promoting peeling of the cells. For example, the device is structured to irradiate the edge of the cell aggregate with the ultrasonic beam having a large output level, meanwhile to irradiate the area other than the edge with the ultrasonic beam having a small output level. The edge recognition unit is also structured to recognize a lift-up status of the cell aggregate, and to make adjustment such as increasing the output level of the ultrasonic beam, for an area without causing the lift-up. Accordingly, this embodiment can efficiently peel the cells, from the outside of the culture vessel in a non-contact manner. With the thus-structured cell culture apparatus equipped with the cell peeling device of this embodiment, the cell peeling step may be automatized, and improved in the throughput.

This embodiment typically discloses the structures below.

The cell peeling device according to this embodiment is a device aimed at peeling cells being cultured while being adhered to the culture vessel, by transmitting ultrasonic wave. The device includes an ultrasonic probe, a move mechanism, a voltage application unit, an edge recognition unit, an edge status database, a probe trajectory calculation unit, and a beam adjustment calculation unit. The ultrasonic probe has a piezoelectric element structured to generate the ultrasonic wave under applied voltage, given in the form of ultrasonic beam. The move mechanism moves the ultrasonic probe, making it possible to change relative position between the ultrasonic probe and the culture vessel. The voltage application unit is connected to the piezoelectric element, and applies voltage to the piezoelectric element. The edge recognition unit recognizes an edge of the cell aggregate in the culture vessel. The edge status database holds coordinate data of the edge of the cell aggregate recognized by the edge recognition unit. The probe trajectory calculation unit calculates a trajectory of the ultrasonic probe, with reference to the status and coordinates of the edge of the cell aggregate, obtained by the edge recognition unit. The beam adjustment calculation unit determines transmission conditions of the ultrasonic wave, with reference to the edge status obtained by the edge recognition unit. Note that the cell peeling device of this embodiment may have a plurality of ultrasonic probes which are independently controlled.

### First Embodiment

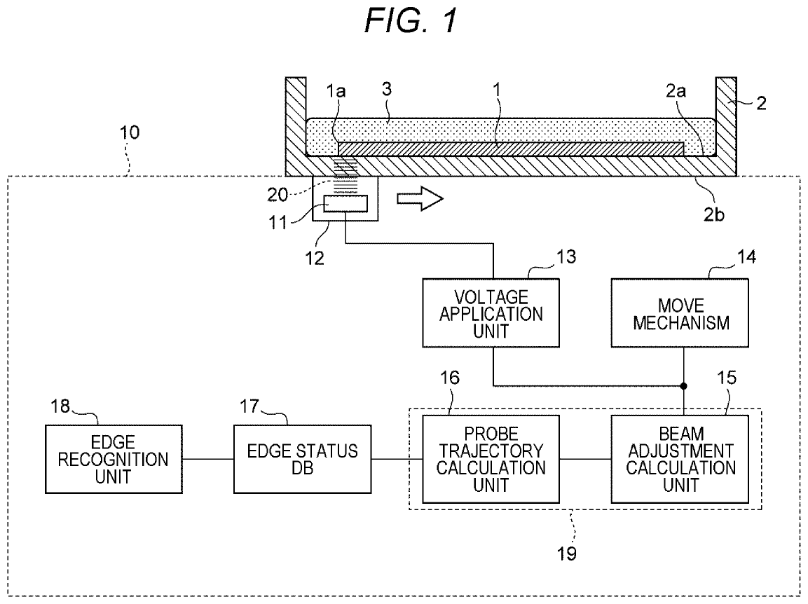

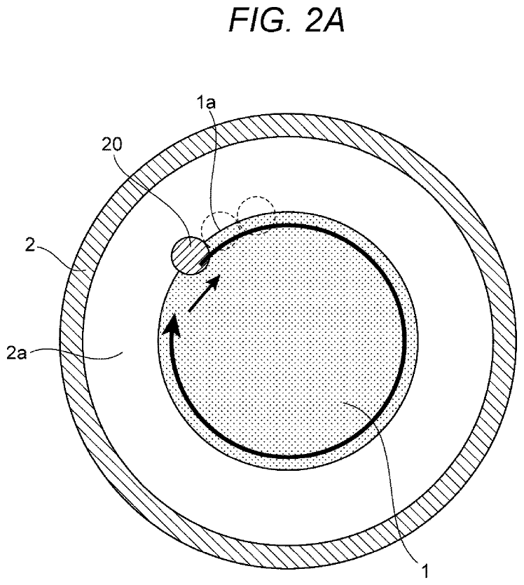

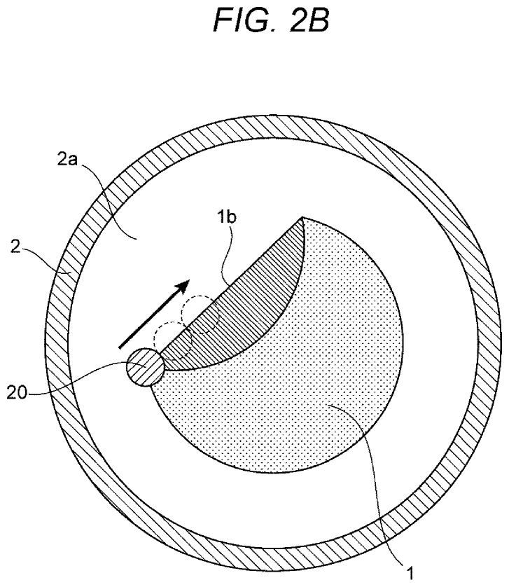

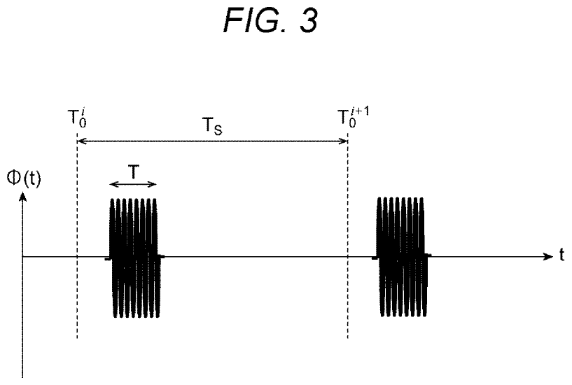

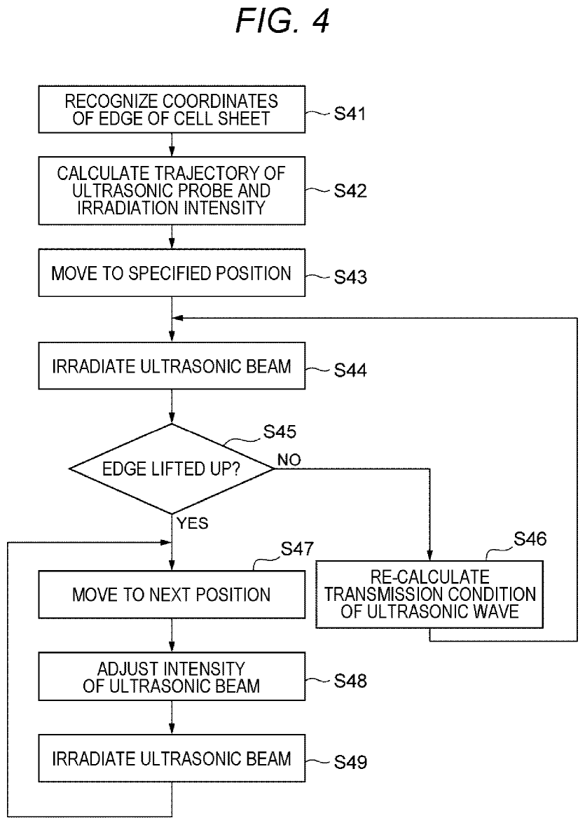

A cell peeling device and a cell peeling method according to a first embodiment will be described with reference to FIGS. 1 to 4. FIG. 1 is a schematic structural diagram of a cell peeling device according to a first embodiment, FIGS. 2A and 2B are schematic diagrams illustrating exemplary moving routes of an ultrasonic beam, FIG. 3 is an explanatory diagram illustrating an exemplary electric signal, and FIG. 4 is a flowchart illustrating details of the cell peeling method according to the first embodiment.

As illustrated in FIG. 1, a cell peeling device 10 irradiates a cell aggregate 1, kept immersed in a culture solution 3 such as a culture medium inside a culture vessel 2, with an ultrasonic beam 20, thereby peeling the cell aggregate 1 from the culture vessel 2. The cell aggregate 1, given typically in the form of cell sheet or organoid, adheres to a bottom inner face 2a of the culture vessel 2.

The cell peeling device 10 has an ultrasonic probe 12 arranged in contact with a bottom outer face 2b of the culture vessel 2, and a voltage application unit 13 structured to apply voltage to a piezoelectric element 11 of the ultrasonic probe 12. The cell peeling device 10 also has a move mechanism 14 structured to move the ultrasonic probe 12 along the bottom outer face 2b of the culture vessel 2 while kept contacted with the bottom outer face 2b, a beam adjustment calculation unit 15 structured to adjust condition of voltage applied to the voltage application unit 13, and a probe trajectory calculation unit 16 structured to calculate a trajectory of the ultrasonic probe 12 moved by the move mechanism 14. The cell peeling device 10 further has an edge recognition unit 18 capable of recognizing an edge 1a of a cell aggregate 1, and an edge status database (DB) 17 that stores the shape and coordinates of the edge 1a of the cell aggregate 1 recognized by the edge recognition unit 18.

The ultrasonic probe 12 irradiates the cell aggregate 1 with the ultrasonic beam 20 having a beam diameter smaller than the size of the cell aggregate 1, from the bottom outer face 2b of the culture vessel 2. The ultrasonic probe 12 typically has a plurality of piezoelectric elements 11 each having a flat plate shape. Each ultrasonic probe 12 has a ground electrode on the upper face side of the piezoelectric element 11, that is, on the culture vessel 2 side, and has a protective film adhered to the upper face side of the ground electrode. The ultrasonic probe 12 is arranged, with the protective film kept contacted with the bottom outer face 2b of the culture vessel 2. The protective film is a thin film formed of metal or resin.

The ultrasonic probe 12 also has a signal electrode through which voltage is applied to the piezoelectric element 11. The piezoelectric element usable herein may have a shape of circle, ellipse, rectangle and so forth, but not limited thereto. A connection point of the signal electrode and a signal wire is covered with a resin, and thus fixed.

The piezoelectric element 11 is formed typically with use of ceramic such as lead zirconate titanate and lead titanate, material such as zinc oxide and lithium niobate, or composite (composite material). The ground electrode and the signal electrode are formed typically with use of material such as gold, silver, copper, platinum, titanium, or aluminum.

The voltage application unit 13 can apply a voltage to the piezoelectric element 11, typically with an electric signal exemplified in FIG. 3. This is an exemplary time waveform, with time t plotted on the abscissa, and with voltage o applied to the piezoelectric element plotted on the ordinate. The voltage application unit 13 applies voltage to the piezoelectric element 11, at a predetermined period Is over a predetermined duration T, as illustrated in FIG. 3.

The move mechanism 14 is a mechanism structured to cause relative movement between the ultrasonic probe 12 and the culture vessel 2. The ultrasonic probe 12 in this embodiment is structured so as to move the ultrasonic probe 12 along the bottom outer face 2b of the culture vessel 2, and along the trajectory calculated by the probe trajectory calculation unit 16. The move mechanism 14 may be constituted by any of known article such as X-Y axis moving table.

The edge status database 17 holds (stores) data regarding the shape and coordinates of the edge of the cell aggregate 1 recognized by the edge recognition unit 18. The edge status database 17 also stores control data of the ultrasonic wave necessary for peeling, such as the size, intensity and transmission time of the ultrasonic beam 20, depending on the type and status of the cultured cell.

The edge recognition unit 18 applicable herein may be any of ordinary cell observation apparatuses, such as optical camera, optical microscope, or phase contrast microscope. The edge recognition unit 18 observes data regarding the shape or the coordinates of the edge of the cell aggregate 1, and stores them in an edge status database 17. The edge may be recognized by image processing with use of an observed cell image. The edge recognition unit 18 may acquire data such as the shape of the cell aggregate 1 and the coordinates of the edge from an external equipment, rather than measuring them. The edge recognition unit 18 may also recognize the lift-up status of the cell aggregate 1.

The beam adjustment calculation unit 15 and the probe trajectory calculation unit 16 are structured to calculate a trajectory along which the ultrasonic probe 12 moves from the edge 1a of the cell aggregate 1, and an output level of the ultrasonic beam 20 at any point on the trajectory, thereby controlling the move mechanism 14 and the ultrasonic probe 12. The beam adjustment calculation unit 15 and the probe trajectory calculation unit 16 constitute a control unit described in the claims.

The probe trajectory calculation unit 16 can freely set the trajectory of the ultrasonic beam 20. The probe trajectory calculation unit 16 calculates a trajectory of the ultrasonic beam 20, along which the ultrasonic beam 20 is irradiated on the boundary between a lift-up area and the adhered area of the cell aggregate 1 after lifting up the edge 1a, where the lift-up area is formed by initially irradiating the ultrasonic beam 20 on the edge 1a recognized by the edge recognition unit 18 before irradiation of the ultrasonic beam 20.

The probe trajectory calculation unit 16 completes, before starting the peeling, calculation of the trajectory along which the entire cell aggregate 1 is peeled from the culture vessel 2. The move mechanism 14 may only move the ultrasonic probe 12 along the trajectory calculated before starting the peeling, thus reducing load for calculation by the beam adjustment calculation unit 15.

Based on the coordinates of the edge of the cell aggregate 1 stored in the edge status database 17, the probe trajectory calculation unit 16 calculates the trajectory along which the ultrasonic beam 20 is first irradiated on the edge of the cell aggregate 1 to cause lift-up at the point of irradiation, and the amount of shift toward a next specified position, typically so as to achieve the trajectory illustrated in FIGS. 2A and 2B. Alternatively, the probe trajectory calculation unit 16 may also calculate the trajectory, while detecting the lift-up status of the cell aggregate 1, and checking the boundary between the adhered area and the lift-up area. The probe trajectory calculation unit 16 in this case calculates the coordinates of the next point of irradiation, and calculates the amount of shift from the current coordinates. The move mechanism 14 moves the ultrasonic probe 12 by such amount of shift.

As illustrated in FIG. 2A, the calculation initially takes place to yield a trajectory, along which the ultrasonic beam 20 can move to lift up the edge 1a of the cell aggregate 1 from the culture vessel 2. The calculation herein takes place so as to make the ultrasonic beam 20 trace a spiral trajectory that swirls from the outer edge of the cell aggregate 1 towards the center. The spiral trajectory may alternatively be calculated so as to make a part of a previously-given outer trajectory and a part of a newly-given inner trajectory overlapped. This can prevent a blank of peeling of the cell aggregate 1.

FIG. 2B illustrates the cell aggregate 1 whose edge 1a has already been lifted up partially, as a result of irradiation with the ultrasonic beam. The probe trajectory calculation unit 16 calculates the amount of shift of the ultrasonic beam 20 to the next specified position, so that the beam will be irradiated progressively along the boundary 1b between the already lifted-up area and the non-lifted-up area of the cell aggregate 1.

Alternatively, the probe trajectory calculation unit 16 may calculate a trajectory having a so-called meandering pattern (not illustrated) along which the cell aggregate 1 may be scanned from the edge, typically in such manner that the ultrasonic beam 20 linearly moves from any desired edge of the cell aggregate 1, and upon arrival at the outer circumference of the cell aggregate 1, jumps to the next row to linearly move backwards.

If there are a plurality of ultrasonic probes 12, the trajectory may be calculated so as to enable irradiation of the ultrasonic beam from each of two or more edges of the cell aggregate 1. In an exemplary case where there is a pair of ultrasonic probes 12, the trajectory may be calculated to have a so-called meandering pattern, along which the probes move from one edge and from the other edge of the cell aggregate 1 in a gradually approaching direction. Alternatively again, the trajectory may be calculated so as to allow a plurality of ultrasonic probes 12, arranged in a line, to move along linearly from a plurality of edges on one side towards a plurality of edges on the other side of the cell aggregate 1.

The beam adjustment calculation unit 15 reads, for example, the shape, adhesion status, and spatial distribution (coordinates) of the edge of a desired cell aggregate 1 being cultured, from the data stored in the edge status database 17. Next, the beam adjustment calculation unit 15 calculates an output of the ultrasonic beam 20, that is, a voltage signal and intensity applied to the ultrasonic probe 12, with reference to the adhesion status of the cell aggregate 1 being cultured. The probe trajectory calculation unit 16 calculates a position of the ultrasonic probe 12 relative to the culture vessel 2. The beam adjustment calculation unit 15 provides the voltage application unit 13 with a condition of the voltage signal to be applied to the ultrasonic probe 12, and provides the move mechanism 14 with a condition of the amount of shift.

The control unit 19, having the beam adjustment calculation unit 15 and the probe trajectory calculation unit 16, may be constituted typically by a computer equipped with a processor such as central processing unit (CPU) or graphics processing unit (GPU), and a memory.

With the CPU structured to load and execute a computer program stored in the memory, it now becomes possible to implement, on the software basis, the calculation function, that is, the functions of the beam adjustment calculation unit 15 and the probe trajectory calculation unit 16 in the control unit 19. Alternatively, a part of or all of the functions may be implemented by hardware. For example, a circuit may be designed to implement the functions, by using a custom IC such as application specific integrated circuit (ASIC), or a programmable IC such as field-programmable gate array (FPGA).

Next, the cell peeling method of this embodiment will be described, while referring to the flowchart in FIG. 4. Note that this embodiment will be described while exemplifying a method of peeling a cell sheet, which is the cell aggregate 1, progressively from the edge towards the inside, but not limited thereto.

As illustrated in FIG. 4, first, the edge recognition unit 18 recognizes the coordinates of the edge of the cell aggregate 1 being cultured (edge recognition step), measures the shape of the cell sheet, the coordinates of the edge and the like, and stores the results in the edge status database 17 (S41).

The probe trajectory calculation unit 16 reads the coordinates of the edge of the cell sheet, relative to the origin of the cell sheet (typically given by the center of the culture vessel 2) stored in the edge status database 17. Next, the probe trajectory calculation unit 16 calculates a trajectory of irradiation point of the ultrasonic beam 20, along which the ultrasonic beam 20 can trace the edge with full coverage, and then can swirl toward the center. Upon or in parallel with the calculation by the probe trajectory calculation unit 16, the beam adjustment calculation unit 15 calculates the condition for the voltage applied to the piezoelectric element 11 of the ultrasonic probe 12, for the thus read coordinates of the edge of the cell sheet, while referring to the intensity of the ultrasonic beam 20 set according to the cell type, the number of culture days of the cell, and the like (S42: ultrasonic adjustment step).

Next, the move mechanism 14 changes the relative position between the ultrasonic probe 12 and the culture vessel 2. More specifically, the move mechanism 14 moves the ultrasonic probe 12 to a specified position, so as to align the center of the ultrasonic beam 20 with a point (origin) of the initial ultrasonic transmission (S43: moving step).

Next, the voltage application unit 13 applies voltage to the piezoelectric element 11 of the ultrasonic probe 12 to generate the ultrasonic wave, thereby causing irradiation of the ultrasonic beam 20 (S44: transmission step).

After irradiation with the ultrasonic beam 20, the edge recognition unit 18 checks whether or not the edge of the cell sheet is lifted up from the culture vessel 2 (S45).

If the edge of the cell sheet was found to lift up from the culture vessel 2 (YES in S45), the move mechanism 14 moves the ultrasonic probe 12 to the next specified position calculated by the probe trajectory calculation unit 16 (S47).

Once the edge of the cell sheet was lifted up, the beam adjustment calculation unit 15 adjusts the output level of the ultrasonic beam 20 (S48). The intensity of the ultrasonic beam herein will be made smaller than the intensity with which the ultrasonic beam 20 was irradiated on the edge, which was the initial irradiation point. Next, the voltage application unit 13 applies voltage to the piezoelectric element 11 of the ultrasonic probe 12, to cause irradiation of the ultrasonic beam 20 (S49).

On the other hand, if the edge of the cell sheet was not found to be lifted up from the culture vessel 2 (NO in S45), the beam adjustment calculation unit 15 recalculates the transmission conditions of the ultrasonic wave, such as the intensity and the transmission time of the ultrasonic beam 20 (S46). Thereafter, the voltage application unit 13 irradiates the ultrasonic beam 20 again under the recalculated conditions (return to S44).

In this embodiment, whether or not the cell sheet was lifted up from the culture vessel 2 is checked upon irradiation of the cell sheet with the ultrasonic beam 20, and if not, the conditions of the ultrasonic beam 20 are recalculated immediately, under which the ultrasonic beam 20 is irradiated again on the same point. The conditions of the ultrasonic beam 20 may be recalculated, for example, so as to elongate the transmission time of the ultrasonic beam 20, or to increase the transmission output.

After irradiating the entire cell sheet with the ultrasonic beam 20, and being confirmed by the edge recognition unit 18 that the entire cell sheet was lifted up, the cell peeling process for one cell sheet comes to the end.

If there is a plurality of cell sheets in one culture vessel 2, or if a cell sheet is also cultured in any other well on a multi-well plate, the move mechanism 14 moves the ultrasonic probe 12 to the next position, that is, any other cell sheet in one culture vessel 2, or any other well, and the aforementioned series of steps of S41 to S49 is repeated to peel the cell sheets from the culture vessel 2.

As described above, the cell peeling device 10 and the cell peeling method of this embodiment can efficiently peel the cell aggregate 1 from the culture vessel 2, in a non-contact manner by irradiating the ultrasonic beam 20 to the cell aggregate 1 being cultured.

The cell peeling device 10 of this embodiment recognizes the edge of the cell aggregate 1, initially irradiates the edge 1a of the cell aggregate 1 with the ultrasonic beam, and starts peeling from the edge 1a of the cell aggregate 1. Thereafter, the device irradiates the cell aggregate 1 with the ultrasonic beam, progressively along the boundary 1b between the already lifted-up area and the non-lifted-up area of the cell aggregate 1, finally causing complete peeling of the cell aggregate 1 from the bottom inner face 2a of the culture vessel 2.

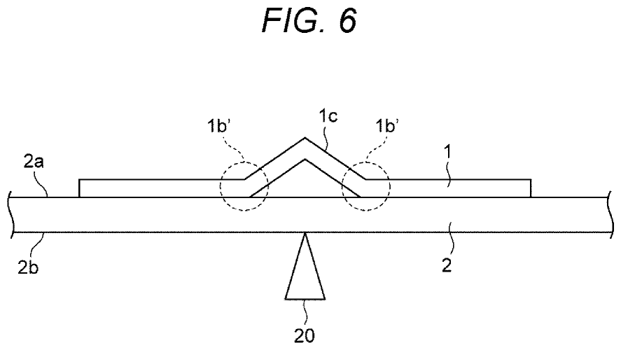

Referring now to FIG. 6 illustrating Comparative Example, an effort of peeling the cell aggregate 1 initially from a center area 1c would break or damage the cells, since both sides of the lifted-up area remain adhered and fixed on the bottom inner face 2a, so that the lift-up thus exerts tensile stress on a boundary 1b′ of the cell aggregate.

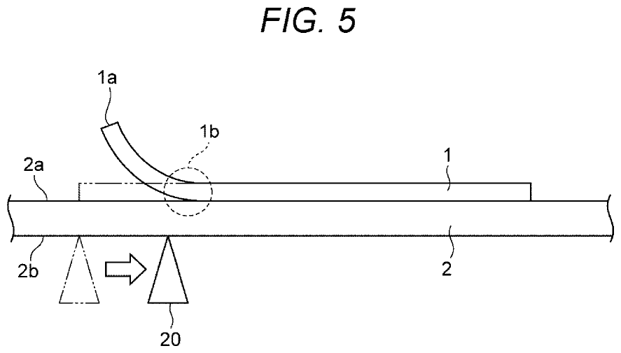

In contrast, the cell peeling device 10 of this embodiment initially irradiates the edge 1a of the cell aggregate 1 with the ultrasonic beam 20, as illustrated in FIG. 5, so as to peel the cell aggregate 1, having been adhered to the bottom inner face 2a of the culture vessel 2, progressively from the edge. Since one side of the peeled area can remain as a free edge, so that no tensile stress would act on the boundary 1b between the lift-up area and the adhered area, causing no risk of breaking or damaging the cells. The cells can therefore be peeled from the vessel at high speed in a non-contact manner.

In the cell peeling device 10 of this embodiment, the beam adjustment calculation unit 15 changes the transmission conditions of the ultrasonic wave transmitted from the ultrasonic probe 12, depending on whether or not the transmission point of the ultrasonic wave directed to the cell aggregate 1 is directed to the edge 1a of the cell aggregate 1. If the transmission point of the ultrasonic wave directed to the cell aggregate 1 is directed to anywhere other than the edge 1a of the cell aggregate 1, the beam adjustment calculation unit 15 reduces the intensity of the ultrasonic beam as compared with the intensity when directed to the edge 1a. For example, the ultrasonic probe 12 sets the output level of the ultrasonic beam to be irradiated on the center area 1c of the cell aggregate 1, lower than that directed to the edge 1a of the cell aggregate 1. This enables rapid and efficient peeling of the cell aggregate 1 from the bottom inner face 2a of the culture vessel 2, without damaging the cells.

This embodiment has described a method structured to confirm presence or absence of lift-up of the cell aggregate 1 being cultured for every irradiation point of the ultrasonic beam 20, and upon confirmation, to move the ultrasonic probe 12 to the next position. The present invention is, however, not limited thereto. For example, the beam adjustment calculation unit 15 may calculate the intensity, transmission time, and coordinates of the ultrasonic beam 20 with reference to the edge profile of the cell aggregate 1 read from the edge status database 17, and may preliminarily prepare plan data for ultrasonic transmission that enables irradiation of the ultrasonic beam 20 over the entire region of the cell sheet. Next, a desired ultrasonic beam 20 may be irradiated on a desired point according to the plan data, with the aid of the voltage application unit 13 and the move mechanism 14, and upon completion of such initial irradiation of the ultrasonic beam 20 according to the plan data, the edge recognition unit 18 may check presence or absence of lift-up of the cell aggregate 1. Next, the plan data for ultrasonic transmission may be prepared again for a point that still remains adhered, and the ultrasonic beam 20 may be transmitted to entirely peel the cell aggregate 1 (cell sheet).

### Second Embodiment

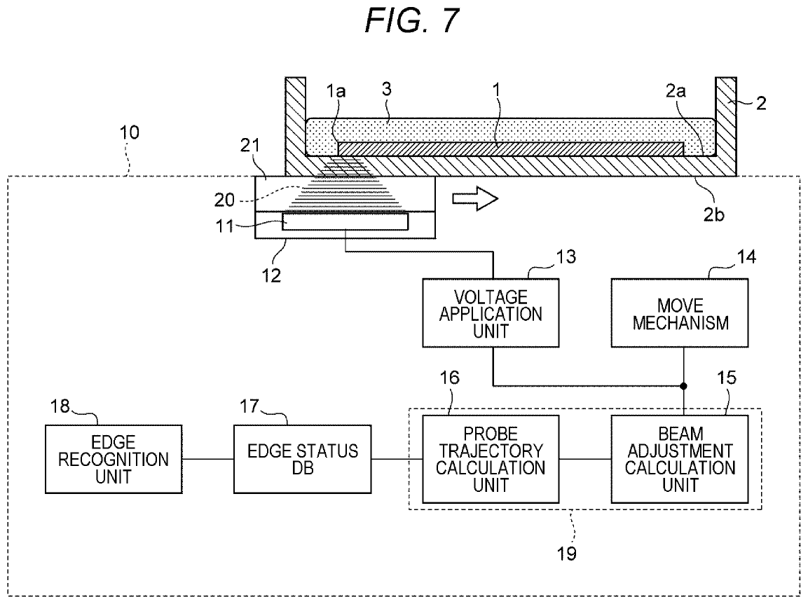

Next, the cell peeling device 10 and the cell peeling method according to a second embodiment will be described with reference to FIG. 7. FIG. 7 is a schematic structural diagram of the cell peeling device 10 according to the second embodiment. Description herein will be focused mainly on differences from the first embodiment.

This embodiment will describe an example of the cell peeling device, in which the size (beam diameter) of the ultrasonic beam on the adhesion surface between the culture vessel and the cell aggregate is variable. Change of the ultrasonic beam size can control the intensity of the ultrasonic beam per area on the adhesion face between the culture vessel and the cell aggregate, and can control the irradiation range, or peeling range, per irradiation of the ultrasonic beam, thus enabling efficient and rapid peeling.

The ultrasonic beam size may be varied with a beam variable mechanism 21, typically by the method below. An acoustic lens, arranged between the ultrasonic probe and the culture vessel, can form an ultrasonic beam with a focal point fallen on any desired point in the direction of transmission axis of the ultrasonic wave. The sizes and focal length of the acoustic lens may be varied. Alternatively, if the ultrasonic probe has a plurality of piezoelectric elements, all of, or a part of the piezoelectric elements may be selected for voltage application. Alternatively, a desired length of delay may be given to the voltage to be applied to the individual piezoelectric elements of the ultrasonic probe, thereby varying the focal position. Alternatively, the frequency of the applied voltage may be varied. Alternatively, the distance of the ultrasonic probe from the adhesion face between the culture vessel and the cells may be varied.

The ultrasonic probe 12 and the beam variable mechanism 21 form the ultrasonic beam focused on a predetermined point. The ultrasonic wave will become strong at a focal position where the ultrasonic wave converges, meanwhile will become weak at an off-focal position where the ultrasonic wave does not converge. The just-focused and thus intensified ultrasonic beam may be irradiated on a predetermined position for a prolonged time, by increasing the number of waves or application period of voltage. This can enhance an effect of peeling the cells having been adhered to the inner wall face of the culture vessel. The methods for controlling the ultrasonic beam size, although having been thus described, are not limited to the aforementioned methods.

An acoustic lens will be described as the beam variable mechanism 21 in this example. Note that the beam variable mechanism 21 is not limited thereto. The acoustic lens has an effect of converging the ultrasonic wave, incident thereon in parallel, to a specific point. The ultrasonic wave, generated by the piezoelectric element 11 under voltage application, passes through the acoustic lens, to form a focal point at a specific point. With this, the ultrasonic wave may be converged even on a point having been less likely to be focused without the acoustic lens. A plurality of acoustic lenses may be provided. With this, a plurality of focal positions may be set. The beam adjustment calculation unit 15 in this case refers to an edge status obtained by the edge recognition unit 18, and determines the transmission conditions of the ultrasonic beam including convergence condition of ultrasonic wave with the aid of the acoustic lens. The transmission conditions are determined typically including the irradiation range and intensity of ultrasonic wave from the ultrasonic probe 12, and characteristics of the acoustic lens to be used.

As described previously and illustrated in FIG. 5, the size of the ultrasonic beam 20 is variable with the acoustic lens such as a Fresnel lens or a concave lens, arranged between the ultrasonic probe 12 and the culture vessel 2. The cell peeling device 10 can vary the size of the ultrasonic beam 20 (beam diameter), by varying the frequency of the voltage to be applied to the piezoelectric element 11 according to the characteristics of the acoustic lens to be used, thereby converging the ultrasonic wave on a desired point.

With use of a Fresnel lens as the acoustic lens, it becomes easier to vary the focal point of the ultrasonic beam 20 and the size of the ultrasonic beam 20, as a result of changing the frequency of the voltage to be applied to the piezoelectric element 11. The voltage application unit 13 in this case applies voltage, with frequency modulation, to the piezoelectric element 11. The beam adjustment calculation unit 15 refers to an edge status of the cell aggregate obtained by the edge recognition unit 18, and determines the transmission conditions of the ultrasonic beam including characteristics of the Fresnel lens and the modulated frequency. The transmission conditions are determined typically including the irradiation range and intensity of ultrasonic wave from the ultrasonic probe 12, and the modulated frequency.

This embodiment is different from the first embodiment only in that the ultrasonic beam 20 from the piezoelectric element 11 is irradiated on a specified position by converging ultrasonic wave through the acoustic lens, while leaving the flow of the cell peeling method unchanged from the first embodiment. The cell peeling method according to the second embodiment will not therefore be described here.

Note however that, in the cell peeling method according to the second embodiment, the peeling planning step (beam adjustment calculation unit 15) determines the conditions of the voltage to be applied by the voltage application unit 13, while considering the number of use and characteristics of the acoustic lenses (how to converge the ultrasonic beam, for example), and the like. For example on the adhesion face, the beam diameter at the edge of the cell aggregate 1 may be reduced than the irradiation spot in anywhere other than the edge.

When a Fresnel lens is used as the beam variable mechanism 21 in the cell peeling method according to the second embodiment, the beam adjustment calculating unit 15 determines the size of the ultrasonic beam 20 to be transmitted through the Fresnel lens, following the conditions suited to peeling of the cell aggregate 1. The beam adjustment calculation unit 15 in this case determines the conditions including not only voltage to be applied to the piezoelectric element 11 of the ultrasonic probe 12 (delay time, etc.), but also frequency to be modulated.

Having detailed the embodiments of the present invention, the present invention is not limited to the aforementioned embodiments, instead allowing various design changes without departing from the spirit of the present invention described in the claims. For example, the aforementioned embodiments have been detailed for easy understanding of the present invention, and are not always limited to those that contain all of the aforementioned structures. A part of a structure of a certain embodiment may be replaced with a structure of another embodiment, and a structure of any other embodiment may be added to a structure of a certain embodiment. Moreover, a part of a structure of the individual embodiments may be allowed for addition, deletion, or replacement of any other structure.

### REFERENCE SIGNS LIST

1 cell aggregate
1a edge
1b boundary
2 culture vessel
2a bottom inner face
2b bottom outer face
3 culture liquid
10 cell peeling device
11 piezoelectric element
12 ultrasonic probe
13 voltage application unit
14 move mechanism
15 beam adjustment calculation unit
16 probe trajectory calculation unit
17 edge status database
18 edge recognition unit
19 control unit
20 ultrasonic beam
21 beam variable mechanism

1. A cell peeling device that peels a cell aggregate from a culture vessel, the cell peeling device comprising:
at least one ultrasonic probe structured to irradiate the cell aggregate with ultrasonic beam having a beam diameter smaller than a size of the cell aggregate, from an outside of the culture vessel;
at least one move mechanism structured to change relative position between the culture vessel and the ultrasonic probe;
an edge recognition unit capable of recognizing an edge of the cell aggregate; and
a control unit structured to control the move mechanism and the ultrasonic probe, by calculating a trajectory along which the ultrasonic probe moves from the edge of the cell aggregate, and an output level of the ultrasonic beam at a point on the trajectory.

2. The cell peeling device according to claim 1, wherein
the control unit is structured to set the output level of the ultrasonic beam irradiated from the ultrasonic probe at the edge of the cell aggregate, to a level higher than the output level of the ultrasonic beam irradiated from the ultrasonic probe at a center part of the cell aggregate.

3. The cell peeling device according to claim 1, further comprising a beam variable mechanism structured to focus ultrasonic wave of the ultrasonic beam.

4. The cell peeling device according to claim 3, wherein
the beam variable mechanism comprises one or more acoustic lenses structured to focus the ultrasonic wave, and
the control unit is structured to determine a transmission condition of the ultrasonic beam, including a focusing condition of the ultrasonic wave by the acoustic lenses.

5. The cell peeling device according to claim 3, wherein
the beam variable mechanism includes a Fresnel lens structured to change a focal point of the ultrasonic wave, and
the control unit is structured to apply, by frequency modulation, voltage to a piezoelectric element of the ultrasonic probe, and to determine the transmission condition of the ultrasonic beam including the Fresnel lens and the modulated frequency with reference to the edge status of the cell aggregate obtained by the edge recognition unit.

6. The cell peeling device according to claim 3, wherein
the beam variable mechanism is capable of changing a beam diameter of the ultrasonic beam, and is structured to reduce the beam diameter at the edge of the cell aggregate, on a contact face between the culture vessel and the cell aggregate, as compared with an irradiation spot in anywhere other than the edge.

7. The cell peeling device according to claim 1, wherein
the control unit is structured to irradiate the ultrasonic beam, after lifting up the edge recognized by the edge recognition unit before irradiation of the ultrasonic beam, on a boundary between a lift-up area and an adhered area of the cell aggregate.

8. The cell peeling device according to claim 1, wherein
the edge recognition unit includes at least one of an optical camera, an optical microscope, or a phase contrast microscope.

9. The cell peeling device according to claim 1, comprising two or more ultrasonic probes and two or more move mechanisms, and
the device being structured to irradiate the ultrasonic beam from each of two or more edges of the cell aggregate recognized by the edge recognition unit.

10. A cell peeling method for peeling a cell aggregate from a culture vessel having cultured cells adhered thereto, the method comprising:
an edge recognition step of recognizing an edge of the cell aggregate;
an ultrasonic adjustment step of calculating a trajectory of an ultrasonic probe and intensity of an ultrasonic beam with reference to coordinates of the edge of the cell aggregate obtained in the edge recognition step;
a moving step of moving the ultrasonic probe to a specified position determined in the ultrasonic adjustment step; and
a transmission step of applying voltage to a piezoelectric element included in the ultrasonic probe to generate ultrasonic wave, and to transmit an ultrasonic beam.
